(19) United States
(12) Patent Application Publication
(10) Pub. No.: US 2026/0115252 A1
(43) Pub. Date: Apr. 30, 2026

(54) Secretoglobins for Suppression of Antibody Responses

(71) Applicant: APC Research Assets LLC, Rockville, MD (US)

(72) Inventor: Aprile L Pilon-Clayton, Rockville, MD (US)

(21) Appl. No.: 18/930,867

(22) Filed: Oct. 29, 2024

(51) Int. Cl. A61K 38/17; A61P 37/06

(52) U.S. Cl. CPC A61K 38/1709; A61P 37/06

(57) ABSTRACT

A method of use of a synthetic SCGB, and compositions thereof, to prevent the development or reduce the development of antibodies against a foreign antigen or set of antigens in the subject that receives the antigen(s) is provided. A synthetic SCGB being recombinant human CC10 protein is provided. The foreign antigen(s) is/are a cell, tissue or organ from a donor subject is administered, introduced, or transplanted into a recipient subject, a lung transplant, transplanted kidney, heart, liver, hematopoietic cell, or any other tissue or organ, antigen(s) is/are from human or non-human origin, purified therapeutic proteins or other drugs, food or drink, self-antigen or an allergen.

### CROSS-REFERENCE TO RELATED APPLICATIONS

This application claims benefit of and priority to U.S. Provisional Application No. 63/337,032, filed Apr. 29, 2022, the disclosure of which is incorporated herein by reference in its entirety.

### BACKGROUND

The development of antibodies against foreign, non-self antigens is a component of the acquired immune response that is essential to host defense during infection. The antigens that primarily enable the immune system to distinguish cells that are self from foreign or non-self are encoded by the MHC and are called MHC1 antigens, a subset of which are called human leukocyte antigens (HLAs). MHC1 antigens are found on all nucleated cells except red blood cells. When foreign, non-self MHC antigens are detected in either transplanted cells, tissues, or organs, or in cell- or tissue-based therapies, or other biologic therapies, the recipient immune system is activated and generates a cell-mediated immune response as well as an antibody against the foreign, non-self MHC antigens, leading to rejection of the transplanted cells or inactivation of the cell-based or biologic therapy.

An acquired immune response against a biologic therapy such as a synthetic or recombinant peptide or protein or a complex containing proteins and/or lipids and/or carbohydrates, typically consists of an antibody response. Such antibody responses are termed anti-drug antibodies (ADA).

Immune responses called autoimmune responses can also arise against self-antigens and cause autoimmune disease. However, the self antigens that generate autoimmune responses and auto-antibodies are typically not MHC antigens but rather are specific proteins, lipids, and/or carbohydrates, or combinations thereof.

Secretoglobins, and SCGB1A1 in particular, have been characterized as “immunomodulatory” proteins. In some studies, SCGB1A1 demonstrated the ability to suppress the development and activity of certain types of T-cells, particularly with respect to the development of allergy and asthma and the cytotoxic activity of NK cells against tumor cells. However, until this work, no SCGB has been shown to suppress the development of an antibody against an antigen. The decrease in titers of circulating DSA in rhCC10-treated animals was, therefore, surprising and provides new applications in the use of rhCC10 to mitigate development of antibodies against non-self antigens introduced into the body for therapeutic, medical, cosmetic, or other purposes.

### SUMMARY OF THE INVENTION

SCGB suppresses the development of an antibody against an antigen. The decrease in titers of circulating DSA in rhCC10-treated animals provides new applications in the use of rhCC10 to mitigate development of antibodies against non-self antigens introduced into the body for therapeutic, medical, cosmetic, or other purposes as further detailed herein.

### BRIEF DESCRIPTION OF THE DRAWINGS

FIG. 1: Histopathology of lung sections from recipient mice treated with Vehicle (placebo) and rhCC10 (1.2 mg/kg) using the 3T-OLTx model. Data from four different lung transplants for each treatment is shown. The lung sections were stained with trichrome and shown at 10× magnification.

FIG. 2: Analysis of transplanted lung tissue. Panel A shows Club cell counts for the Vehicle (V)-treated group and the CC10-treated group. Panel B shows hydroxyproline content in micrograms per milligram of tissue for the Vehicle (V)-treated group and the CC10-treated group. The red line indicates historical mean for untreated lung transplants that develop OB at POD 16. N=4/group. The star (*) indicates a p-value of <0.05 using the Student's T-test.

FIG. 3: Analysis of immunological outcomes. Panel A shows quantitated Donor-Specific Antibodies (IgM in plasma) measured as mean fluorescence index (MFI) in a titering assay. Panel B quantitates the number of IL-17A+CD4+ cells as a percent of CD4+ T cells in lung transplants measured by fluorescence-activated cell sorting (FACS). Panel C quantitates the number of IFN-gamma+CD8+ cells as a percent of CD8+ T cells in lung transplants measured by FACS. The red line indicates historical mean for untreated lung transplants that develop OB at POD 16. N=8/group. The triple star (***) indicates a p-value of less than 0.01 using the unpaired Student's T-test.

### DETAILED DESCRIPTION

In another aspect of the invention, the SCGB preparation prevents development of antibodies against a specific antigen or combination of antigens in an individual when administered at the same time as the antigen(s) or any time after the individual is exposed to the antigen(s).

In transplant applications, the SCGB may be perfused into or added to the donor tissue donor prior to or after the extraction of the donor tissue, or it may be administered to the recipient of the donor tissue concurrently with the transplant or at any time after the transplant in order to prevent development of donor-specific antibodies (DSA).

In drug therapies that may or do elicit development of anti-drug antibodies (ADA), the SCGB may be administered at the same time as, or any time after, the drug therapy in order to prevent development of ADA. Examples of drug therapies that elicit ADA include enzyme replacement therapy, antibiotics and other complex small molecule drugs, and any biologic therapy such as synthetic or native purified proteins, lipids, nucleic acids; exosomes, liposomes, micelles, antibiotics, anti-fungals, anti-virals, and all cell-based therapies).

In autoimmune diseases, antibodies may be formed against self-antigens, called auto-antibodies (AA), that may harm the afflicted individual. The administration of a SCGB may be used to prevent development of an auto-antibody, or reduce the titers of existing auto-antibodies in a susceptible or afflicted individual.

In another aspect of the invention, the antigen that elicits the antibody response is an allergen such as house dust mite, danders from a cat or other animal, or tree or grass pollen.

The administration of a SCGB includes not only administering a purified synthetic SCGB protein, but also includes the administration of a biologic entity that functions as a delivery vehicle and is capable of synthesizing or expressing a SCGB protein (such as a cell engineered to express a SCGB gene) or a virus or phage that is capable of delivering a synthetic SCGB gene to a cell in a susceptible or afflicted individual that then expresses the SCGB in a susceptible or afflicted individual. The materials and methods to create such biologic delivery vehicles for therapeutic proteins, vaccines, and other treatments are well-known. Administration of such biologic delivery vehicles that effectively deliver SCGB proteins to prevent development and/or reduce titers of DSA, ADA, AA, or other harmful types of antibodies are yet another aspect of the invention.

In another embodiment, a nucleic acid (DNA, RNA, or mimetic thereof) encoding the human SCGB1A1 protein is included in a biologic treatment comprising a cell, cell-derived vesicle or exosome, synthetic liposome or vesicle, phage, mRNA or other vaccine, phage, virus, bacterium or other microbe, from which the SCGB1A1 protein is intended to be expressed as a component of the treatment. The treatment is administered to a patient, SCGB1A1 protein is expressed by the treatment, and suppresses the development of antibodies against any component of the treatment.

In another embodiment, a nucleic acid (DNA, RNA, or mimetic thereof) encoding the human SCGB1A1 protein is included in a biologic treatment comprising a delivery vehicle and another nucleic acid encoding an auto-antigen for the purpose of suppressing the development of auto-antibodies against the auto-antigen. More specifically, this embodiment may contain a nucleic acid encoding both the auto-antigen and SCGB1A1 intended to be co-expressed for the purpose of suppressing the titers of the auto-antibodies.

### Example 1: rhSCGB1A1/CC10 in Lung Transplant Rejection

Lung transplantation (LTx) is a life-saving procedure for patients with end-stage lung diseases, such as cystic fibrosis (CF). However, the median survival after LTx remains poor, around 5-6 years, primarily limited by chronic lung allograft dysfunction (CLAD). CLAD is the clinical manifestation of chronic rejection, is characterized by airway and parenchymal fibrosis, and the development of donor-specific antibodies and antibodies against lung antigens. Repeated injuries to the allograft, such as infection, acute rejection, and toxic inhalational exposures also contribute to the development of CLAD. In particular, infectious non-alloimmune stress that causes epithelial injury is linked to CLAD [70].

Orthotopic lung transplant (OLTx) between mismatched HLA (human leukocyte antigens) of donor and recipient mice models the immunological mechanisms underlying the lung inflammation and fibrosis found in human CLAD. A component of these immunological mechanisms involves the development of DSA and various T- and B-cells. The mouse OLTx technique was first reported in 2007 [2] and is now an established approach to study mechanisms of injury and evaluate potential therapies in orthotopic lung transplant [3]. We used a newer OLTx model in which conditional Club cell ablation is achieved using genetically-engineered mouse strains to promote lung and immunologic phenotypes that closely resemble human chronic lung transplant rejection, obliterative bronchiolitis (OB), and chronic lung allograft dysfunction (CLAD) (described in [1] and referred to as 3T-OLTx model). In the 3T-OLTx model, lungs from mice with the FVB haplotype are transplanted into mice with the B6 haplotype. The FVB mice are a triple transgenic (3T) strain that carries 3 genes that mediate conditional Club cell depletion, including; 1) a reverse tetracycline activator gene driven by the Club cell secretory protein (CC10) promoter, 2) a Cre recombinase gene under control of the reverse tetracycline activator, and 3) a lox-P activated diphtheria toxin A gene (DT-A). Ingestion of doxycycline (DOX) by 3T mice induces Cre-mediated recombination of the lox-P DT-A locus to enable diphtheria toxin expression specifically in CC10-expressing cells, resulting in their depletion and injury to bronchiolar epithelium [4]. For these transplant experiments, 3T mice were extensively backcrossed to FVB and B6 backgrounds to create allogeneic (3T FVB) donor lungs that were transplanted into B6 recipient mice (3T FVB>B6) [1]. In order to induce tolerance to the donor FVB lung, antibody against CD40L is administered to the B6 recipient on the day of transplant and antibody against CTLA4 is administered 2 days after transplant (post-operative day 2, “POD 2”). Then, in order to simulate epithelial injury that breaks the tolerance, DOX is administered on POD 7-9 which eliminates ˜80% of the Club cells in the donor lung. This Club cell depletion using DOX-induced expression of DT-A simulates infection-mediated injury that occurs in Pseudomonas aeruginosa (PA) infection, a known risk factor for CLAD development [5], which releases a PA toxin that, like DT-A, inhibits eEF2-dependent protein synthesis to kill airway epithelial cells to promote CLAD [6]. If DOX is not administered, or control lung allografts that lack the diphtheria toxin gene, then the established tolerance is maintained.

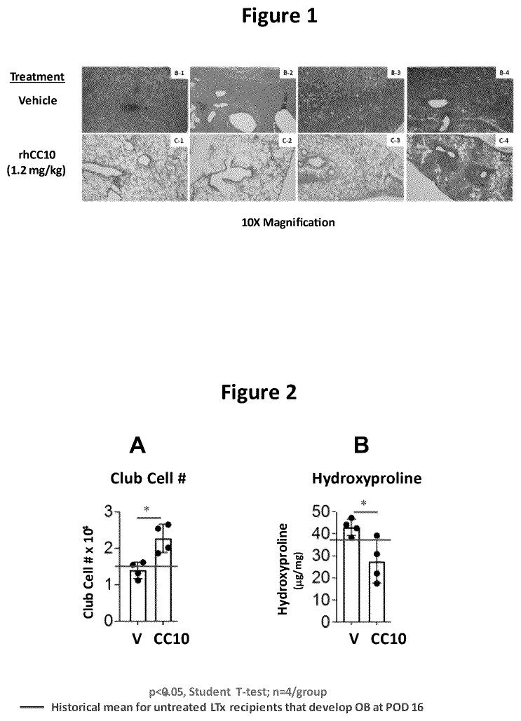

The 3T FVB>B6 recipients received daily intratracheal administration of recombinant human protein (rhSCGB1A1 or rhCC10) at a dose of 1.2 mg/kg/day starting on POD 8 and ending on POD 15. Mice were then sacrificed on POD 16 and lungs, plasma, and whole blood were collected for analysis using previously described methods [1]. Lung histopathology (trichrome staining) showed that fibrosis and OB were significantly reduced in rhCC10-treated lungs (FIG. 1). Club cell counts and hydroxyproline content were similarly improved by rhCC10 treatment (FIG. 2). The decrease in pulmonary fibrosis assessed by lung histopathology and hydroxyproline content are similar to two published studies in murine models of pulmonary fibrosis induced by bone marrow transplant [7, 8] and in a model of bleomycin-induced pulmonary fibrosis in mice [9]. However, this is the first report that recombinant human CC10 protein directly increased Club cell counts following significant lung epithelial injury and acute loss of ˜80% of Club cells.

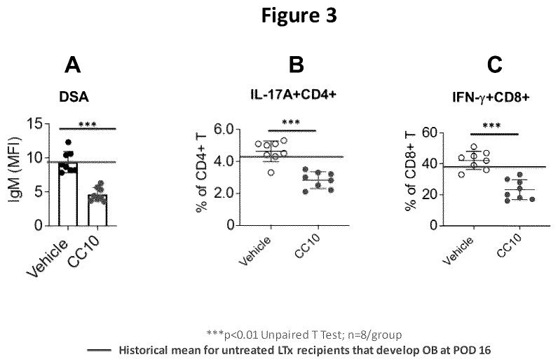

There was also a significant impact of rhCC10 on immunological endpoints, including reducing DSA, and IL-17A+CD4+ and IFN-gamma+CD8+ T-cells (FIG. 3). In humans, the presence of CLAD correlates with elevated DSA, IL-17A+CD4+, IFN-gamma+CD8+, and total T-cell numbers in the lungs [10, 11]. This is the first evidence that CC10 administration can impact the development of and/or titer of any type of antibody. This is also the first evidence that CC10 can impact the development of and/or number of IFN-gamma+CD8+ T-cells in the lungs.

In summary, key features of the 3T-OLTx model are representative of the immunological and histopathological manifestations of CLAD in humans, including: 1) formation of DSA and T-cell responses, and 2) airway injury, inflammation, fibrosis, and OB.

It is possible and desirable to construct the following embodiments by combining the features disclosed prior and hereafter:
1. In a first embodiment, a method of use of a synthetic SCGB to prevent the development or reduce the development of antibodies against a foreign antigen or set of antigens in the subject that receives the antigen(s) is provided.
2. In a 2^{nd} embodiment further adding one or more features of the 1^{st} embodiment, the synthetic SCGB is recombinant human CC10 protein.
3. In a 3^{rd} embodiment further adding one or more features of the 1^{st} and/or 2^{nd} embodiments, the foreign antigen(s) is/are a cell, tissue or organ from a donor subject is administered, introduced, or transplanted into a recipient subject.
4. In a 4^{th} embodiment further adding one or more of the features of the 1^{st} through 3^{rd} embodiments, the foreign antigen is a lung transplant.
5. In a 5^{th} embodiment further adding one or more of the features of the 1^{st} through 4^{th} embodiments, the foreign antigen is a transplanted kidney, heart, liver, hematopoietic cell, or any other tissue or organ.
6. In a 6^{th} embodiment further adding one or more of the features of the 1^{st} through 5^{th} embodiments, the foreign antigen(s) is/are from human or non-human origin.
7. In a 7^{th} embodiment further adding one or more of the features of the 1^{st} through 6^{th} embodiments, the foreign antigen(s) is/are a purified therapeutic proteins or other drugs.
8. In an 8^{th} embodiment further adding one or more of the features of the 1^{st} through 7^{th} embodiments, the foreign antigen(s) is/are a food or drink.
9. In a 9^{th} embodiment further adding one or more of the features of the 1^{st} through 8^{th} embodiments, the antigen is self-antigen.
10. In a 10^{th} embodiment further adding one or more of the features of the 1^{st} through 9^{th} embodiments, the antigen is an allergen.
11. In an 11^{th} embodiment, a composition of matter comprised of a synthetic SCGB administered in combination with, contemporaneous with, or any time after a foreign antigen or group of foreign antigens, to a recipient subject, that does not elicit DSA in the recipient subject is provided.
12. In a 12^{th} embodiment further adding one or more of the features of the 11^{th} embodiment, the foreign antigen is a cell, tissue, or organ originating from a donor subject.
13. In a 13^{th} embodiment further adding one or more of the features of the 11^{th} and/or 12^{th} embodiments, the foreign antigen is purified therapeutic proteins or other drugs.
14. In a 14^{th} embodiment further adding one or more of the features of the 11^{th} through 13^{th} embodiments, the foreign antigen is a food or drink.
15. In a 15^{th} embodiment further adding one or more of the features of the 11^{th} through 14^{th} embodiments, the foreign antigen is a self-antigen.
16. In a 16^{th} embodiment further adding one or more of the features of the 11^{th} through 15^{th} embodiments, the foreign antigen is an allergen.
17. In a 17^{th} embodiment further adding one or more of the features of the 11^{th} through 16^{th} embodiments, the synthetic SCGB is CC10.

### Definitions

Biologic delivery vehicle: Any nucleic acid, endosome, micelle or vesicle, cell, microbe, phage or virus, or tissue that is engineered with the intent to deliver a specific protein or set of proteins through expression of the specific protein(s) and is administered to a subject by any route of administration.

Synthetic SCGB: Any formulation or preparation of a SCGB protein or biologic delivery vehicle that expresses a SCGB that can be administered to a mammal for any therapeutic application.

Foreign antigen: Any substance that originates from outside the body of a subject that elicits or can elicit an immune response from the subject resulting in the formation of an antibody or an immune cell response that binds to the substance or any component thereof.

Self-antigen: Any substance that originates from inside the body of a subject that elicits or can elicit an immune response from the subject resulting in the formation of an antibody or an immune cell response that binds to the substance or any component thereof.

Donor-specific antibody (DSA): Any class of antibody (IgM, IgA, IgG, IgE, IgD and all subtypes) that develops in a recipient subject into which a foreign antigen, cell, tissue, or organ that derived from a donor subject was administered, introduced, or transplanted and that binds to the foreign antigen(s). DSA recognize donor HLA and/or MHC antigens in the graft.

Anti-drug antibody (ADA): Any class of antibody (IgM, IgA, IgG, IgE, IgD and all subtypes) that develops in a subject into which a drug was administered, introduced, or transplanted that binds to the drug.

Auto-antibody: Any class of antibody (IgM, IgA, IgG, IgE, IgD and all subtypes) that develops in a subject that binds to any self-antigen(s).

Club cells (also known as Clara cells): Non-ciliated airway epithelial cells found primarily in the small airways of the lungs that secrete many proteins and substances essential for normal lung physiology, including the SCGB1A1/CC10 protein.

Major Histocompatibility Complex (MHC): The MHC is a large polygenic locus on vertebrate DNA that encode cell surface proteins that enable the recognition of self-antigens by the cells of the adaptive immune system such as T-cells and leukocytes. Three types of MHC antigens are expressed in humans. MHC1 are highly conserved with respect to structure and functions and are expressed on essentially all nucleated human cells (except red blood cells). MHC2 are highly conserved with respect to structure and functions and are expressed on antigen-presenting cells, lung and gut epithelial cells, some endothelial cells, thymic epithelial cells, and B cells. MHC3 antigens are structurally and functionally diverse and are expressed by liver cells, some white blood cells, and other cells.

Human Leukocyte Antigen (HLA): Proteins encoded by the MHC that are displayed on the surface of leukocytes and other cells. HLAs are a subset of the MHC antigens and are unique to humans.

Obliterative bronchiolitis (OB): A type of pulmonary fibrosis in which the small airways become occluded by the replacement of normal airway epithelial cells with fibroblasts, mucus, and extracellular matrix. In the context of lung transplant, OB is caused primarily by the immune response against the donor HLAs.

Secretoglobin (SCGB): A small globular, multimeric protein that is secreted, each monomer of which holds a four helical bundle secondary structure, belonging to the secretoglobin family of proteins.

Secretoglobin 1A1 (SCGB1A1), also known as CC10, CC16, CCSP, uteroglobin (UG, UTG), urine protein-1 (UP-1), and other names, is the most well-known member of the SCGB family of proteins

### REFERENCES

1. Liu L, Liao F, Scozzi D, Furuya Y, Pugh K N, Hachem R, Chen D L, Cano M, Green J M, Krupnick A S, Kreisel D, Perl A K T, Huang H J, Brody S L, A E Gelman. An obligatory role for club cells in preventing obliterative bronchiolitis in lung transplants. JCI Insight. 2019 Apr. 16; 5 (9): e124732.
2. Okazaki M, Krupnick A S, Kornfeld C G, Lai J M, Ritter J H, Richardson S B, Huang H J, Das N A, Patterson G A, Gelman A E, Kreisel D. A mouse model of orthotopic vascularized aerated lung transplantation. Am J Transplant. 2007 June; 7 (6): 1672-9.
3. Lama V N, Belperio J A, Christie J D, El-Chemaly S, Fishbein M C, Gelman A E, Hancock W W, Keshavjee S, Kreisel D, Laubach V E, Looney M R, McDyer J F, Mohanakumar T, Shilling R A, Panoskaltsis-Mortari A, Wilkes D S, Eu J P, Nicolls M R. Models of Lung Transplant Research: a consensus statement from the National Heart, Lung, and Blood Institute workshop. JCI Insight. 2017 May 4; 2 (9): e93121.
4. Perl A K, Riethmacher D, Whitsett J A. Conditional depletion of airway progenitor cells induces peribronchiolar fibrosis. Am J Respir Crit Care Med. 2011 Feb. 15; 183 (4): 511-21.
5. Gregson A L. Infectious Triggers of Chronic Lung Allograft Dysfunction. Curr Infect Dis Rep. 2016 July; 18 (7): 21.
6. Iglewski B H, Liu P V, Kabat D. Mechanism of action of Pseudomonas aeruginosa exotoxin Aiadenosine diphosphate-ribosylation of mammalian elongation factor 2 in vitro and in vivo. Infect Immun. 1977 January; 15 (1): 138-44.
7. Bustos, M. L., M. Mura, D. Hwang, O. Ludkovski, A. P. Wong, A. Keating and T. K. Waddell (2015). “Depletion of bone marrow CCSP-expressing cells delays airway regeneration.” Mol Ther 23 (3): 561-569.
8. Wendt, C., K. Tram, A. Price, K. England, A. Stiehm and A. Panoskaltsis-Mortari (2013). “Club cell secretory protein improves survival in a murine obliterative bronchiolitis model.” Am J Physiol Lung Cell Mol Physiol 305 (9): L642-650.
9. Cai, Y., M. E. Winn, J. K. Zehmer, W. K. Gillette, J. T. Lubkowski, A. L. Pilon and S. Kimura (2014). “Preclinical evaluation of human secretoglobin 3A2 in mouse models of lung development and fibrosis.” Am J Physiol Lung Cell Mol Physiol 306 (1): L10-22.
10. Hodge, S., et al. Increased levels of T cell granzyme b in bronchiolitis obliterans syndrome are not suppressed adequately by current immunosuppressive regimens. Clin Exp Immunol 158, 230-236 (2009).
11. Burlingham, W. J., et al. IL-17-dependent cellular immunity to collagen type V predisposes to obliterative bronchiolitis in human lung transplants. J Clin Invest 117, 3498-3506 (2007).

1. A method of use of a synthetic SCGB to prevent the development or reduce the development of antibodies against a foreign antigen or set of antigens in the subject that receives the antigen(s).

2. The method of claim 1 in which the synthetic SCGB is recombinant human CC10 protein.

3. The method of claim 1 in which the foreign antigen(s) is/are a cell, tissue or organ from a donor subject is administered, introduced, or transplanted into a recipient subject.

4. The method of claim 1 in which the foreign antigen is a lung transplant.

5. The method of claim 1 in which the foreign antigen is a transplanted kidney, heart, liver, hematopoietic cell, or any other tissue or organ.

6. The method of claim 1 in which the foreign antigen(s) is/are from human or non-human origin.

7. The method of claim 1 in which the foreign antigen(s) is/are a purified therapeutic proteins or other drugs.

8. The method of claim 1 in which the foreign antigen(s) is/are a food or drink.

9. The method of claim 1 in which the antigen is self-antigen.

10. The method of claim 1 in which the antigen is an allergen.

11. A composition of matter comprised of a synthetic SCGB administered in combination with, contemporaneous with, or any time after a foreign antigen or group of foreign antigens, to a recipient subject, that does not elicit DSA in the recipient subject.

12. The composition of claim 11 in which the foreign antigen is a cell, tissue, or organ originating from a donor subject.

13. The composition of claim 11 in which the foreign antigen is purified therapeutic proteins or other drugs.

14. The composition of claim 11 in which the foreign antigen is a food or drink.

15. The composition of claim 11 in which the foreign antigen is a self-antigen.

16. The composition of claim 11 in which the foreign antigen is an allergen.

17. The composition of claim 11 in which the synthetic SCGB is CC10.
